(19) United States
(12) Patent Application Publication
(10) Pub. No.: US 2018/0085330 A1
(43) Pub. Date: Mar. 29, 2018

(54) Dose Adjustment

(71) Applicant: AbbVie Inc., Waukegan, IL (US)

(72) Inventors: Sandeep Dutta, Lincolnshire, IL (US); Matthew Kosloski, Wilmette, IL (US); Wei Liu, Mundelein, IL (US)

(73) Assignee: AbbVie Inc., North Chicago, IL (US)

(21) Appl. No.: 15/713,137

(22) Filed: Sep. 22, 2017

Related U.S. Application Data
(60) Provisional application No. 62/398,724, filed on Sep. 23, 2016.
(60) Provisional application No. 62/510,936, filed on May 25, 2017.

(51) Int. Cl. A61K 31/192; A61K 38/13; A61K 31/22

(52) U.S. Cl. CPC A61K 31/192; A61K 31/22; A61K 38/13

(57) ABSTRACT

This application features dose adjustment for drugs co-administered with glecaprevir and pibrentasvir.

### RELATED APPLICATION

This application claims the benefit of U.S. Provisional Ser. No. 62/398,724, filed Sep. 23, 2016 and claims the benefit of U.S. Provisional Ser. No. 62/510,936, filed May 25, 2017. The contents of each application are incorporated herein by reference.

### FIELD OF THE TECHNOLOGY

This application relates to dose adjustment for drugs coadministered with glecaprevir and pibrentasvir.

### BACKGROUND

Treatment of chronic Hepatitis C virus (HCV) infection with interferon-free regimens of direct-acting antivirals (DAA) has become the new standard of care, offering higher efficacy, better tolerability and safety, and shorter treatment durations than previous interferon and ribavirin based therapies. Glecaprevir

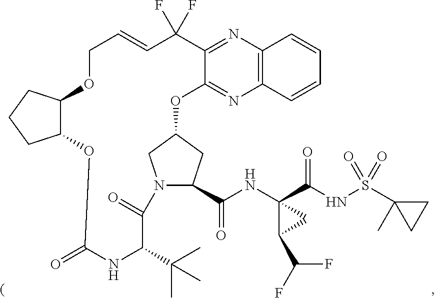 [SMILES: CC(C)(C)[C@@H]1NC(=O)O[C@@H]2CCC[C@H]2OC/C=C/C(F)(F)c2nc3ccccc3nc2O[C@@H]2C[C@@H](C(=O)N[C@]3(C(=O)NS(=O)(=O)C4(C)CC4)C[C@H]3C(F)F)N(C2)C1=O]

CAS No. 1365970-03-1), a protease inhibitor, and pibrentasvir

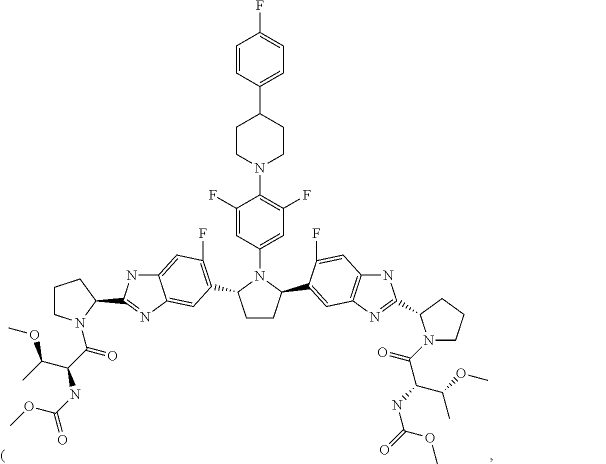

CAS No. 1353900-92-1), a NS5A inhibitor, are potent anti-HCV agents. The all oral, ribavirin-free combination of glecaprevir and pibrentasvir has been recently approved by Food and Drug Agency, European Medical Agency and Health Canada as MAVYRET® or MAVIRET® for treatment of chronic HCV. The Prescribing Information for MAVYRET and European Public Assessment Report are both incorporated herein by reference in its entirety. The treatment duration is as short as 8, 12 or 16 weeks. The glecaprevir and pibrentasvir combination has been shown to be effective against numerous HCV genotypes including genotypes 1, 2, 3, 4, 5 and 6. However, it is not readily known whether the glecaprevir and pibrentasvir combination, when co-administered with other drugs, would lead to undesired drug-drug interactions, thereby requiring dosing adjustment of the other drugs.

### SUMMARY OF THE INVENTION

This application relates to dose adjustment for drugs co-administered with glecaprevir and pibrentasvir. In one embodiment, the invention provides a method of treating patients infected with hepatitis C virus having an independent co-morbid condition. In this method, the patients are co-administered glecaprevir and pibrentasvir once daily, together with a concomitant drug for treating the co-morbid condition. The drug is selected from a group consisting of drugs that are substrates of Organic Anion Transporting Polypeptide (OATP), P-glycoprotein (P-gp) and Breast Cancer Resistance Protein (BRCP). In this method, the established dose of the drug is dose-adjusted prior to or simultaneously when administering glecaprevir and pibrentasvir to the patients. In another embodiment, the glecaprevir is administered 300 mg once a day and the pibrentasvir is administered 120 mg once a day. Further, in another embodiment, the glecaprevir and the pibrentasvir is administered for a duration of 8, 12 or 16 weeks, or as described in the Prescribing Information for MAVYRET® or MAVIRET®. In another embodiment, the patient has a HCV genotype of 1, 2, 3, 4, 5 or 6 or the patient is treatment naïve, treatment experienced or has cirrhosis.

In another embodiment, the method teaches that the drug is selected from a group consisting of digoxin, pravastatin, rosuvastatin, fluvastatin, pitavastatin and cyclosporine. Preferably the established dose of the drug is dose-adjusted based on independently following selection of embodiments wherein the digoxin dose is reduced by 50% of the established dose or wherein the pravastatin dose is reduced by 50% of the established dose or wherein the rosuvastatin dose is no more than 10 mg per day or wherein the cyclosporine dose is no more than 100 mg per day or wherein the fluvastatin or pitavastatin dose is reduced to lowest approved dose or lowest necessary dose, if higher doses are required. Further, preferably, when administering the glecaprevir and the pibrentasvir combination, the following drugs are independently not recommended or independently contraindicated for a concomitant treatment such as atazanavir, rifampin, carbamazepine, hypericum perforatum (St. John's Wort), efavirenz, ethinyl estradiol containing medications, darunavir, lopinavir, ritonavir, atorvastatin, lovastatin or simvastatin.

In yet another embodiment, the present invention provides a method of treating patients infected with hepatitis C virus having an independent co-morbid condition. This method comprises co-administering to said patients glecaprevir and pibrentasvir once daily, together with a concomitant drug for treating the co-morbid condition. In this method, the established dose of the drug is dose-adjusted prior to or simultaneously when administering glecaprevir and pibrentasvir to the patients. In another embodiment, the glecaprevir is administered 300 mg once a day and the pibrentasvir is administered 120 mg once a day. Further, in another embodiment, the glecaprevir and the pibrentasvir is administered for a duration of 8, 12 or 16 weeks, or as described in the Prescribing Information for MAVYRET® or MAVIRET®. In another embodiment, the patient has a HCV genotype of 1, 2, 3, 4, 5 or 6 or the patient is treatment naïve, treatment experienced or has cirrhosis.

In another embodiment, the method teaches that the drug is selected from a group consisting of digoxin, pravastatin, rosuvastatin, fluvastatin, pitavastatin and cyclosporine. Preferably the established dose of the drug is dose-adjusted based on independently following selection of embodiments wherein the digoxin dose is reduced by 50% of the established dose or wherein the pravastatin dose is reduced by 50% of the established dose or wherein the rosuvastatin dose is no more than 10 mg per day or wherein the cyclosporine dose is no more than 100 mg per day or wherein the fluvastatin or pitavastatin dose is reduced to lowest approved dose or lowest necessary dose, if higher doses are required. Further, preferably, when administering the glecaprevir and the pibrentasvir combination, the following drugs are independently not recommended or independently contraindicated for a concomitant treatment such as atazanavir, rifampin, carbamazepine, hypericum perforatum (St. John's Wort), efavirenz, ethinyl estradiol containing medications, darunavir, lopinavir, ritonavir, atorvastatin, lovastatin or simvastatin.

A further embodiment of the invention provides a method of treating patients infected with hepatitis C virus having an independent co-morbid condition. This method comprises co-administering to the patients glecaprevir and pibrentasvir once daily, together with a concomitant drug for treating the co-morbid condition. In this method, the established dose of the drug is dose-adjusted prior to or simultaneously when administering glecaprevir and pibrentasvir to the patients. Preferably in this method, the drug is selected from a group consisting of digoxin, pravastatin, rosuvastatin, fluvastatin, pitavastatin and cyclosporine.

In this method, the drug dose-adjustment is selected from a group consisting of (a) the digoxin dose is reduced by 50% of the established dose, (b) the pravastatin dose is reduced by 50% of the established dose, (c) the rosuvastatin dose is no more than 10 mg per day, (d) the cyclosporine dose is no more than 100 mg per day, and (e) the fluvastatin or pitavastatin dose is reduced to lowest approved dose or lowest necessary dose.

In this method, another embodiment provides that the glecaprevir is administered 300 mg once a day and the pibrentasvir is administered 120 mg once a day. Further, in another embodiment, the glecaprevir and the pibrentasvir is administered for a duration of 8, 12 or 16 weeks, or as described in the Prescribing Information for MAVYRET® or MAVIRET®. In another embodiment, the patient has a HCV genotype of 1, 2, 3, 4, 5 or 6 or the patient is treatment naïve, treatment experienced or has cirrhosis.

It should be understood that the above summary and the description and examples are given by way of illustration, not limitation. Therefore, variations such as +/−20% for dose adjustment ranges are within the contemplated ranges of the dose adjustment of the drugs. Various changes and modifications within the scope of the present application will become apparent to those skilled in the art from the present description.

### DETAILED DESCRIPTION

HCV patients sometimes have other co-morbid conditions that may require treatment with other drugs. In medicine, comorbidity is the presence of one or more additional diseases or disorders co-occurring with (that is, concomitant or concurrent with) a primary disease or disorder; in the countable sense of the term, a comorbidity (plural comorbidities) is each additional disorder or disease. When glecaprevir and pibrentasvir are used with other concomitant drugs, dose adjustment may be needed for the other drugs due to drug-drug interactions. Dose adjustment means that an established dose is either increased on decreased such that a desirable plasma concentration Cmax or area under the curve AUC is achieved. Typically, desirable Cmax and AUC are determined by a treating physician or by a Prescribing Information of a given drug. Thus, for example, to dose adjust digoxin, the following process may be followed: first, measure serum digoxin concentrations before initiating glecaprevir and pibrentasvir treatment, then reduce digoxin concentrations by decreasing the dose by approximately 50% or by modifying the dosing frequency. While some drugs may be dose adjusted to desirable Cmax and AUC levels, certain other concomitant drugs are independently contraindicated or independently not recommended altogether, including the group consisting of atazanavir, rifampin, carbamazepine, hypericum perforatum (St. John's Wort), efavirenz, ethinyl estradiol containing medications (such as oral contraceptives), darunavir, lopinavir, ritonavir, atorvastatin, lovastatin and simvastatin. For dose adjustment, an established dose is a dose provided by a physician or as described in a prescribing information or a public assessment report of the drug as approved by a regulatory agency, such as Food and Drug Agency or European Medical Agency, Health Canada and the like.

Glecaprevir and pibrentasvir are inhibitors of P-glycoprotein (P-gp), breast cancer resistance protein (BCRP), and organic anion transporting polypeptide (OATP) 1B1/3. Coadministration with glecaprevir and pibrentasvir may increase plasma concentration of drugs that are substrates of P-gp, BCRP, OATP1B1 or OATP1B3. Glecaprevir and pibrentasvir are weak inhibitors of cytochrome P450 (CYP) 3A, CYP1A2, and uridine glucuronosyltransferase (UGT) 1A1. Significant interactions are not expected when combination is coadministered with substrates of CYP3A, CYP1A2, CYP2C9, CYP2C19, CYP2D6, UGT1A1, or UGT1A4.

Glecaprevir and pibrentasvir are substrates of P-gp and/or BCRP. Glecaprevir is a substrate of OATP1B1/3. Coadministration of glecaprevir and pibrentasvir with drugs that inhibit hepatic P-gp, BCRP, or OATP1B1/3 may increase the plasma concentrations of glecaprevir and/or pibrentasvir.

Coadministration of glecaprevir and pibrentasvir with other concomitant drugs that induce P-gp/CYP3A may decrease glecaprevir and pibrentasvir plasma concentrations.

In one embodiment, the present invention feature methods of treating patients infected with HCV, where the patients are also treated with digoxin. The methods comprise administering glecaprevir and pibrentasvir once daily to the patients, where the digoxin dose is reduced by 50%.

As used herein, any dose reduction of a drug when co-administered with glecaprevir and pibrentasvir is measured relative to the dose that would be normally administered without co-administration of glecaprevir and pibrentasvir. For example, if a patient has been using 300 mcg digoxin once daily (QD) before the initiation of the glecaprevir and pibrentasvir combination therapy, then co-administration of glecaprevir and pibrentasvir requires the reduction of digoxin dose by 50% from 300 mcg once daily.

In another embodiment, the present invention feature methods of treating patients infected with HCV, where the patients are also treated with digoxin. The methods comprise administering 300 mg glecaprevir and 120 mg pibrentasvir once daily to the patients, where the digoxin dose is reduced by 50%.

In yet another embodiment, the present invention feature methods of treating patients infected with HCV, where the patients are also treated with pravastatin. The methods comprise administering glecaprevir and pibrentasvir once daily to the patients, where the pravastatin dose is reduced by 50%.

In yet another embodiment, the present invention feature methods of treating patients infected with HCV, where the patients are also treated with pravastatin. The methods comprise administering 300 mg glecaprevir and 120 mg pibrentasvir once daily to the patients, where the pravastatin dose is reduced by 50%.

In yet another embodiment, the present invention feature methods of treating patients infected with HCV, where the patients are also treated with rosuvastatin and would be treated with rosuvastatin at a dose of more than 10 mg per day when not coadministered with glecaprevir and pibrentasvir. The methods comprise administering glecaprevir and pibrentasvir once daily to the patients, where the rosuvastatin dose is no more than 10 mg per day.

In yet another embodiment, the present invention feature methods of treating patients infected with HCV, where the patients are also treated with rosuvastatin and would be treated with rosuvastatin at a dose of more than 10 mg per day when not coadministered with glecaprevir and pibrentasvir. The methods comprise administering 300 mg glecaprevir and 120 mg pibrentasvir once daily to the patients, where the rosuvastatin dose is no more than 10 mg per day.

In still yet another embodiment, the present invention feature methods of treating patients infected with HCV, where the patients are also treated with cyclosporine. The methods comprise administering glecaprevir and pibrentasvir once daily only to the patients who are on stable doses of cyclosporine of no more than 100 mg per day.

In still yet another embodiment, the present invention feature methods of treating patients infected with HCV, where the patients are also treated with cyclosporine. The methods comprise administering 300 mg glecaprevir and 120 mg pibrentasvir once daily only to the patients who are on stable doses of cyclosporine of no more than 100 mg per day.

In one embodiment, the invention provides a method of treating patients infected with hepatitis C virus having an independent co-morbid condition. In this method, the patients are co-administered glecaprevir and pibrentasvir once daily, together with a concomitant drug for treating the co-morbid condition. The drug is selected from a group consisting of drugs that are substrates of Organic Anion Transporting Polypeptide (OATP), P-glycoprotein (P-gp) and Breast Cancer Resistance Protein (BRCP). In this method, the established dose of the drug is dose-adjusted prior to or simultaneously when administering glecaprevir and pibrentasvir to the patients. In another embodiment, the glecaprevir is administered 300 mg once a day and the pibrentasvir is administered 120 mg once a day. Further, in another embodiment, the glecaprevir and the pibrentasvir is administered for a duration of 8, 12 or 16 weeks, or as described in the Prescribing Information for MAVYRET® or MAVIRET®. In another embodiment, the patient has a HCV genotype of 1, 2, 3, 4, 5 or 6 or the patient is treatment naïve, treatment experienced or has cirrhosis.

In another embodiment, the method teaches that the drug is selected from a group consisting of digoxin, pravastatin, rosuvastatin, fluvastatin, pitavastatin and cyclosporine. Preferably the established dose of the drug is dose-adjusted based on independently following selection of embodiments wherein the digoxin dose is reduced by 50% of the established dose or wherein the pravastatin dose is reduced by 50% of the established dose or wherein the rosuvastatin dose is no more than 10 mg per day or wherein the cyclosporine dose is no more than 100 mg per day or wherein the fluvastatin or pitavastatin dose is reduced to lowest approved dose or lowest necessary dose, if higher doses are required. Further, preferably, when administering the glecaprevir and the pibrentasvir combination, the following drugs are independently not recommended or independently contraindicated for a concomitant treatment such as atazanavir, rifampin, carbamazepine, hypericum perforatum (St. John's Wort), efavirenz, ethinyl estradiol containing medications, darunavir, lopinavir, ritonavir, atorvastatin, lovastatin or simvastatin.

In yet another embodiment, the present invention provides a method of treating patients infected with hepatitis C virus having an independent co-morbid condition. This method comprises co-administering to said patients glecaprevir and pibrentasvir once daily, together with a concomitant drug for treating the co-morbid condition. In this method, the established dose of the drug is dose-adjusted prior to or simultaneously when administering glecaprevir and pibrentasvir to the patients. In another embodiment, the glecaprevir is administered 300 mg once a day and the pibrentasvir is administered 120 mg once a day. Further, in another embodiment, the glecaprevir and the pibrentasvir is administered for a duration of 8, 12 or 16 weeks, or as described in the Prescribing Information for MAVYRET® or MAVIRET®. In another embodiment, the patient has a HCV genotype of 1, 2, 3, 4, 5 or 6 or the patient is treatment naïve, treatment experienced or has cirrhosis.

In another embodiment, the method teaches that the drug is selected from a group consisting of digoxin, pravastatin, rosuvastatin, fluvastatin, pitavastatin and cyclosporine. Preferably the established dose of the drug is dose-adjusted based on independently following selection of embodiments wherein the digoxin dose is reduced by 50% of the established dose or wherein the pravastatin dose is reduced by 50% of the established dose or wherein the rosuvastatin dose is no more than 10 mg per day or wherein the cyclosporine dose is no more than 100 mg per day or wherein the fluvastatin or pitavastatin dose is reduced to lowest approved dose or lowest necessary dose, if higher doses are required. Further, preferably, when administering the glecaprevir and the pibrentasvir combination, the following drugs are independently not recommended or independently contraindicated for a concomitant treatment such as atazanavir, rifampin, carbamazepine, hypericum perforatum (St. John's Wort), efavirenz, ethinyl estradiol containing medications, darunavir, lopinavir, ritonavir, atorvastatin, lovastatin or simvastatin.

A further embodiment of the invention provides a method of treating patients infected with hepatitis C virus having an independent co-morbid condition. This method comprises co-administering to the patients glecaprevir and pibrentasvir once daily, together with a concomitant drug for treating the co-morbid condition. In this method, the established dose of the drug is dose-adjusted prior to or simultaneously when administering glecaprevir and pibrentasvir to the patients. Preferably in this method, the drug is selected from a group consisting of digoxin, pravastatin, rosuvastatin, fluvastatin, pitavastatin and cyclosporine.

In this method, the drug dose-adjustment is selected from a group consisting of (a) the digoxin dose is reduced by 50% of the established dose, (b) the pravastatin dose is reduced by 50% of the established dose, (c) the rosuvastatin dose is no more than 10 mg per day, (d) the cyclosporine dose is no more than 100 mg per day, and (e) the fluvastatin or pitavastatin dose is reduced to lowest approved dose or lowest necessary dose.

In this method, another embodiment provides that the glecaprevir is administered 300 mg once a day and the pibrentasvir is administered 120 mg once a day. Further, in another embodiment, the glecaprevir and the pibrentasvir is administered for a duration of 8, 12 or 16 weeks, or as described in the Prescribing Information for MAVYRET® or MAVIRET®. In another embodiment, the patient has a HCV genotype of 1, 2, 3, 4, 5 or 6 or the patient is treatment naïve, treatment experienced or has cirrhosis.

It should be understood that the above description and the following examples are given by way of illustration, not limitation. Various changes and modifications within the scope of the present application will become apparent to those skilled in the art from the present description.

### Example 1. Established and Other Potential Drug Interaction

Table 1 provides the effect of coadministration of glecaprevir and pibrentasvir on concentrations of concomitant drugs and the effect of concomitant drugs on glecaprevir and pibrentasvir.

TABLE 1
Potentially Significant Drug Interactions Identified in Drug
Interaction Studies that May Require Dose Alteration

| | | |
|---|---|---|
| Concomitant | | |
| Drug Class: | Effect on | |
| Drug Name | Concentration | Clinical Comments |

Antiarrhythmics:

| | | |
|---|---|---|
| Digoxin | ↑ digoxin | Digoxin dose should be |
| | | reduced by 50% when |
| | | coadministered with |
| | | glecaprevir and |
| | | pibrentasvir. |

Anticoagulants:

| | | |
|---|---|---|
| Dabigatran | ↑ dabigatran | If glecaprevir, pibrentasvir |
| etexilate | | and dabigatran etexilate are |
| | | coadministered, refer to the |
| | | dabigatran etexilate |
| | | prescribing information |
| | | for dabigatran etexilate |
| | | dose modifications in |
| | | combination with P-gp |
| | | inhibitors in the |
| | | setting of renal impairment. |

Anticonvulsants:

| | | |
|---|---|---|
| Carbamazepine | ↓ glecaprevir | Coadministration may lead |
| | ↓ pibrentasvir | to reduced therapeutic |
| | | effect of glecaprevir and |
| | | pibrentasvir and is |
| | | not recommended. |

Ethinyl Estradiol-Containing Products:

| | | |
|---|---|---|
| Ethinyl estradiol- | [symbol] glecaprevir | Coadministration of glecaprevir |
| containing | [symbol] pibrentasvir | and pibrentasvir with ethinyl |
| medications such | | estradiol-containing products |
| as combined oral | | may increase the risk of |
| contraceptives | | ALT elevations and is not |
| | | recommended. |

Herbal Products:

| | | |
|---|---|---|
| St. John's Wort | ↓ glecaprevir | Coadministration may lead |
| (hypericum | ↓ pibrentasvir | to reduced therapeutic effect |
| perforatum) | | of glecaprevir and |
| | | pibrentasvir and is not |
| | | recommended. |

HIV-Antiviral Agents:

| | | |
|---|---|---|
| Darunavir | ↑ glecaprevir | Coadministration is not |
| Lopinavir | ↑ pibrentasvir | recommended. |
| Ritonavir | | |
| Efavirenz | ↓ glecaprevir | Coadministration may lead |
| | ↓ pibrentasvir | to reduced therapeutic effect |
| | | of glecaprevir and |
| | | pibrentasvir and is |
| | | not recommended. |

HMG-CoA Reductase Inhibitors:

| | | |
|---|---|---|
| Pravastatin | ↑ pravastatin | Pravastatin dose should be |
| Rosuvastatin | ↑ rosuvastatin | reduced by 50% and |
| | | rosuvastatin dose should not |
| | | exceed 10 mg per day when |
| | | co-administered with glecaprevir |
| | | and pibrentasvir. |
| | | Co-administration may |
| | | significantly increase the |
| | | concentration of rosuvastatin. |
| | | Increased statin concentrations |
| | | may increase the risk of |
| | | myopathy, including |
| | | rhabdomyolysis. |
| | | Rosuvastatin may be |
| | | administered with |
| | | MAVYRET at a dose |
| | | that does not exceed 10 mg. |
| Atorvastatin | ↑ atorvastatin | Concomitant use is not recommended. |
| Lovastatin | ↑ lovastatin | Consider alternative therapies, |
| Simvastatin | ↑ simvastatin | such as pravastatin or rosuvastatin. |
| Fluvastatin | ↑ fluvastatin | Co-administration may |
| Pitavastatin | ↑ pitavastatin | increase the concentrations of |
| | | fluvastatin and pitavastatin. |
| | | Increased statin concentrations |
| | | may increase the risk of myopathy, |
| | | including rhabdomyolysis. |
| | | Use the lowest approved dose |
| | | of fluvastatin or pitavastatin. |
| | | If higher doses are needed, |
| | | use the lowest necessary statin |
| | | dose based on a risk/ |
| | | benefit assessment. |

Immunosuppressants:

| | | |
|---|---|---|
| Cyclosporine | ↑ glecaprevir | Glecaprevir and pibrentasvir |
| | ↑ pibrentasvir | is not recommended for use |
| | | in patients requiring stable |
| | | cyclosporine doses >100 mg |
| | | per day. |

See Clinical Pharmacology Tables 2 and 3
↑ = increase; ↓ = decrease; [symbol] = no effect.

### Example 2. Clinical Pharmacology: Drug Interaction Studies

Drug interaction studies were performed with glecaprevir/pibrentasvir and other drugs that are likely to be coadministered and with drugs commonly used as probes for pharmacokinetic interactions. Tables 2 and 3 summarize the pharmacokinetic effects when glecaprevir/pibrentasvir was coadministered with other drugs which showed potentially clinically relevant changes.

TABLE 1
Drug Interactions: Changes in Pharmacokinetic Parameters of Glecaprevir
or Pibrentasvir in the Presence of Coadministered Drug

| Co- | Regimen of Co- | Regimen of | | | | | | | | |
|---|---|---|---|---|---|---|---|---|---|---|
| administered | administered | GLE/PIB | | | Central Value Ratio (90% CI) | | | | | |
| Drug | Drug (mg) | (mg) | N | DAA | Cₘₐₓ | | AUC | | Cₘᵢₙ | |
| Atazanavir + | 300 + 100 | 300/120 | 12 | GLE | ≥4.06 | (3.15, 5.23) | ≥6.53 | (5.24, 8.14) | ≥14.3 | (9.85, 20.7) |
| ritonavir | once daily | once daily^{a} | | PIB | ≥1.29 | (1.15, 1.45) | ≥1.64 | (1.48, 1.82) | ≥2.29 | (1.95, 2.68) |
| Carbamazepine | 200 | 300/120 | 10 | GLE | 0.33 | (0.27, 0.41) | 0.34 | (0.28, 0.40) | — | |
| | twice daily | single dose | | PIB | 0.50 | (0.42, 0.59) | 0.49 | (0.43, 0.55) | — | |
| Cyclosporine | 100 | 300/120 | 12 | GLE | 1.30 | (0.95, 1.78) | 1.37 | (1.13, 1.66) | 1.34 | (1.12, 1,60) |
| | single dose | once daily | | PIB | [symbol] | | [symbol] | | 1.26 | (1.15, 1.37) |
| | 400 | 300/120 | 11 | GLE | 4.51 | (3.63, 6.05) | 5.08 | (4.11, 6.29) | — | |
| | single dose | single dose | | PIB | [symbol] | | 1.93 | (1.78, 2.09) | — | |
| Darunavir + | 800 + 100 | 300/120 | 8 | GLE | 3.09 | (2.26, 4.20) | 4.97 | (3.62, 6.84) | 8.24 | (4.40, 15.4) |
| ritonavir | once daily | once daily | | PIB | [symbol] | | [symbol] | | 1.66 | (1.25, 2.21) |
| Elvitegravir | 150/150/ | 300/120 | 11 | GLE | 2.50 | (2.08, 3.00) | 3.05 | (2.55, 3.64) | 4.58 | (3.15, 6.65) |
| cobicistat | 200/10 | once daily | | PIB | [symbol] | | 1.57 | (1.39, 1.76) | 1.89 | (1.63, 2.19) |
| emtricitabine/ | once daily | | | | | | | | | |
| tenofovir | | | | | | | | | | |
| alafenamide | | | | | | | | | | |
| Omeprazole | 20 | 300/120 | 9 | GLE | 0.78 | (0.60, 1.00) | 0.71 | (0.58, 0.86) | — | |
| | once daily | single dose | | PIB | [symbol] | | [symbol] | | — | |
| | 40 | 300/120 | 12 | GLE | 0.36 | (0.21, 0.59) | 0.49 | (0.35, 0.68) | — | |
| | once daily | single dose | | PIB | [symbol] | | [symbol] | | — | |
| | (1 hour | | | | | | | | | |
| | before | | | | | | | | | |
| | GLE/PIB) | | | | | | | | | |
| Rifampin | 600 | 300/120 | 12 | GLE | 6.52 | (5.06, 8.41) | 8.55 | (7.01, 10.4) | — | |
| | (first close) | single dose | | PIB | [symbol] | | [symbol] | | — | |
| | 600 | 300/120 | 12 | GLE | 0.14 | (0.11, 0.19) | 0.12 | (0.09, 0.15) | — | |
| | once daily | single dose^{b} | | PIB | 0.17 | (0.14, 0.20) | 0.13 | (0.11, 0.15) | — | |
| Lopinavir/ | 400/100 | 300/120 | 9 | GLE | 2.55 | (1.84, 3.52) | 4.38 | (3.02, 6.36) | 18.6 | (10.4, 33.5) |
| ritonavir | twice daily | once daily | | PIB | 1.40 | (1.17, 1.67) | 2.46 | (2.07, 2.92) | 5.24 | (4.18, 6.58) |

[symbol]

= No change (central value ratio 0.80 to 1.25)
^{a}Effect of atazanavir and ritonavir on the first dose of glecaprevir and pibrentasvir is reported.
^{b}Effect of rifampin on glecaprevir and pibrentasvir 24 hours after final rifampin dose.

TABLE 3
Drug Interactions: Pharmacokinetic Parameters for Coadministered
Drug in the Presence of Combination of Glecaprevir/Pibrentasvir

| Co- | Regimen of Co- | | | | | | | | |
|---|---|---|---|---|---|---|---|---|---|
| administered | administered | Regimen of | | Central Value Ratio (90% CI) | | | | | |
| Drug | Drug (mg) | GLE/PIB (mg) | N | Cₘₐₓ | | AUC | | Cₘᵢₙ | |
| Abacavir | ABC/DTG/3TC | 300/120 | 12 | [symbol] | | [symbol] | | 1.31 | (1.05, 1.63) |
| | 600/50/300 | once daily | | | | | | | |
| | once daily | | | | | | | | |
| Atorvastatin | 10 | 400/120 | 11 | 22.0 | (16.4, 29.6) | 8.28 | (6.06, 11.3) | — | |
| | once daily | once daily | | | | | | | |
| Caffeine | 100 | 300/120 | 12 | [symbol] | | 1.35 | (1.23, 1.48) | — | |
| | single dose | once daily | | | | | | | |
| Dabigatran | Dabigatran | 300/120 | 11 | 2.05 | (1.72, 2.44) | 2.38 | (2.11, 2.70) | — | |
| | etexilate | once daily | | | | | | | |
| | 150 | | | | | | | | |
| | single dose | | | | | | | | |
| Darunavir | DRV + RTV | 300/120 | 12 | 1.30 | (1.21, 1.40) | 1.29 | (1.18, 1.42) | [symbol] | |
| Ritonavir | 800 + 100 | once daily | | 2.03 | (1.78, 2.32) | 1.87 | (1.74, 2.02) | [symbol] | |
| | once daily | | | | | | | | |
| Dextro- | Dextromethorphan | 300/120 | 12 | 0.70 | (0.61, 0.81) | 0.75 | (0.66, 0.85) | — | |
| methorphan | hydrobromide | once daily | | | | | | | |
| | 30 | | | | | | | | |
| | single dose | | | | | | | | |
| Digoxin | 0.5 | 400/120 | 12 | 1.72 | (1.45, 2.04) | 1.48 | (1.40, 1.57) | — | |
| | single dose | once daily | | | | | | | |
| Ethinyl | EE/ | 300/120 | 11 | 1.31 | (1.24, 1.38) | 1.28 | (1.23, 1.32) | 1.38 | (1.25, 1.52) |
| estradiol (EE) | Norgestimate | once daily | | | | | | | |
| Norgestrel | 35 μg/250 μg | | | 1.54 | (1.34, 1.76) | 1.63 | (1.50, 1.76) | 1.75 | (1.62, 1.89) |
| Norgestromin | once daily | | | [symbol] | | 1.44 | (1.34, 1.54) | 1.45 | (1.33, 1.58) |
| Ethinyl | EE/ | 300/120 | 12 | 1.30 | (1.18, 1.44) | 1.40 | (1.33, 1.48) | 1.56 | (1.41, 1.72) |
| estradiol | Levonorgestrel | once daily | | | | | | | |
| Norgestrel | 20 μg/100 μg | | | 1.37 | (1.23, 1.52) | 1.68 | (1.57, 1.80) | 1.77 | (1.58, 1.98) |
| | once daily | | | | | | | | |
| Elvitegravir | EVG/COBI/FTC/ | 300/120 | 12 | 1.36 | (1.24, 1.49) | 1.47 | (1.37, 1.57) | 1.71 | (1.50, 1.95) |
| Tenofovir | TAF 150/ | once daily | | [symbol] | | [symbol] | | [symbol] | |
| | 150/200/10 | | | | | | | | |
| | once daily | | | | | | | | |
| Felodipine | 2.5 | 300/120 | 11 | 1.31 | (1.05, 1.62) | 1.31 | (1.08, 1.58) | — | |
| | single close | once daily | | | | | | | |
| Losartan | 50 | 300/120 | 12 | 2.51 | (2.00, 3.15) | 1.56 | (1.28, 1.89) | — | |
| Losartan | single dose | once daily | | 2.18 | (1.88, 2.53) | [symbol] | | — | |
| carboxylic | | | | | | | | | |
| acid | | | | | | | | | |
| Lovastatin | Lovastatin | 300/120 | 12 | [symbol] | | 1.70 | (1.40, 2.06) | — | |
| Lovastatin | 10 | once daily | | 5.73 | (4.65, 7.07) | 4.10 | (3.45, 4.87) | — | |
| acid | once daily | | | | | | | | |
| Midazolam | 1 | 300/120 | 12 | [symbol] | | 1.27 | (1.11, 1.45) | — | |
| | single dose | once dally | | | | | | | |
| Omeprazole | 20 | 300/120 | 12 | 0.57 | (0.43, 0.75) | 0.79 | (0.70, 0.90) | — | |
| | single dose | once daily | | | | | | | |
| Pravastatin | 10 | 400/120 | 12 | 2.23 | (1.87, 2.65) | 2.30 | (1.91, 2.76) | — | |
| | once daily | once daily | | | | | | | |
| Raltegravir | 400 | 300/120 | 12 | 1.34 | (0.89, 1.98) | 1.47 | (1.15, 1.87) | 2.64 | (1.42, 4.91) |
| | twice daily | once daily | | | | | | | |
| Rilpivirine | 25 | 300/120 | 12 | 2.05 | (1.73, 2.43) | 1.84 | (1.72, 1.98) | 1.77 | (1.59, 1.96) |
| | once daily | once daily | | | | | | | |

| | | | | | | | | |
|---|---|---|---|---|---|---|---|---|
| Rosuvastatin | 5 | 400/120 | 11 | 5.62 | (4.80, 6.59) | 2.15 | (1.88, 2.46) | — |
| | once daily | once daily | | | | | | |
| Simvastatin | Simvastatin | 300/120 | 12 | 1.99 | (1.60, 2.48) | 2.32 | (1.93, 2.79) | — |
| Simvastatin | 5 | once daily | | 10.7 | (7.88, 14.6) | 4.48 | (3.11, 6.46) | — |
| acid | once daily | | | | | | | |
| Sofosbuvir | Sofosbuvir | 400/120 | 8 | 1.66 | (1.23, 1.22) | 2.25 | (1.86, 2.72) | — |

| | | | | | | |
|---|---|---|---|---|---|---|
| GS-331007 | 400 | once daily | [symbol] | [symbol] | 1.85 | (1.67, 2.04) |
| | once daily | | | | | |

| | | | | | | | | |
|---|---|---|---|---|---|---|---|---|
| Tacrolimus | 1 | 300/120 | 10 | 1.50 | (1.24, 1.82) | 1.45 | (1.24, 1.70) | — |
| | single dose | once daily | | | | | | |

| | | | | | | | | |
|---|---|---|---|---|---|---|---|---|
| Tenofovir | EFV/FTC/TDF | 300/120 | 12 | [symbol] | 1.29 | (1.23, 1.35) | 1.38 | (1.31, 1.46) |
| | 300/200/300 | once daily | | | | | | |
| | once daily | | | | | | | |

| | | | | | | | | |
|---|---|---|---|---|---|---|---|---|
| Valsartan | 80 | 300/120 | 12 | 1.36 | (1.17, 1.58) | 1.31 | (1.16, 1.49) | — |
| | single dose | once daily | | | | | | |

[symbol]

= No change (central value ratio 0.80 to 1.25)
3TC—lamivudine;
ABC—abacavir;
COBI—cobicistat;
DRV—darunavir;
DTG—dolutegravir;
EFV—efavirenz;
EVG—elvitegravir;
FTC—emtricitabine;
RTV—ritonavir;
TAF—tenofovir alafenamide;
TDF—tenofovir disoproxil fumarate

### Example 3. Digoxin

This Example assessed pharmacokinetics, safety, and tolerability of multiple doses of glecaprevir and pibrentasvir with a single dose of digoxin. Adult male and female subjects in general good health (N=12) were enrolled in the study. The study design was shown in Table 4.

TABLE 4

| Period 1 | | Period 2 | | |
|---|---|---|---|---|
| Day 1 | | Days 1 to 7 | Day 8 | Days 9 to 12 |
| Digoxin | 10-day | | Digoxin 0.5 mg | |

| | | |
|---|---|---|
| 0.5 mg | Washout | glecaprevir 400 mg QD + |
| | | pibrentasvir 120 mg QD |

Digoxin Cₘₐₓ and AUC_{inf} were 72% and 48% higher, respectively, when coadministered with glecaprevir and pibrentasvir compared to digoxin alone. Estimates of renal clearance and fraction of digoxin eliminated in urine were similar (≦18% difference) with and without glecaprevir and pibrentasvir, suggesting limited inhibition of renal P-gp. Glecaprevir and pibrentasvir exposures were similar (≦16% difference) with and without digoxin.

P-glycoprotein (P-gp) is an efflux transporter expressed in a variety of tissues including the apical membrane of intestinal epithelial cells, renal proximal tubular cells, brain capillary endothelial cells, and the canalicular membrane of hepatocytes. The cardiac glycoside digoxin is often utilized as a probe substrate to clinically evaluate P-gp mediated drug-drug interactions. Glecaprevir and pibrentasvir increased digoxin exposure, suggesting that the glecaprevir and pibrentasvir combination may inhibit P-gp. Based on the results of this study, digoxin dose should be reduced by 50% when coadministered with glecaprevir and pibrentasvir, and patients should be monitored.

### Example 4. Pravastatin, Rosuvastatin or Atorvastatin

This Example assessed pharmacokinetics, safety, and tolerability of multiple doses of glecaprevir and pibrentasvir with multiple doses of pravastatin, rosuvastatin or atorvastatin. Adult male and female subjects in good health (N=12/arm, N=36 total) were enrolled in the study. The study design was shown in Table 5.

TABLE 5

| | Period 1 | | Period 2 | Period 3 |
|---|---|---|---|---|
| Arm 1 | Day 1 | | Days 1 to 3 | Days 1 to 7 |
| | glecaprevir | 8-day | Pravastatin 10 mg QD | |
| | 400 mg + | Washout | | glecaprevir 400 mg |
| | pibrentasvir | | | QD + pibrentasvir |
| | 120 mg | | | 120 mg QD |
| | Period 1 | | Period 2 | Period 3 |
| Arm 2 | Day 1 | | Days 1 to 7 | Days 1 to 7 |
| | glecaprevir | 6-day | Rosuvastatin 5 mg QD | |
| | 400 mg + | Washout | | glecaprevir 400 mg |
| | pibrentasvir | | | QD + pibrentasvir |
| | 120 mg | | | 120 mg QD |
| | Period 1 | | Period 2 | Period 3 |
| Arm 3 | Day 1 | | Days 1 to 7 | Days 1 to 7 |
| | glecaprevir | 7-day | Atorvastatin 10 mg QD | |
| | 400 mg + | Washout | | glecaprevir |
| | pibrentasvir | | | 400 mg QD + |
| | 120 mg | | | pibrentasvir |
| | | | | 120 mg QD |

When coadministered with glecaprevir and pibrentasvir, pravastatin Cₘₐₓ and AUC24 were 2.2- and 2.3-fold, respectively, of pravastatin alone. Glecaprevir exposures were higher (↑59% Cₘₐₓ, ↑44% AUC₂₄) when glecaprevir and pibrentasvir were coadministered with pravastatin than for glecaprevir and pibrentasvir alone; whereas pibrentasvir exposures were similar (≦24% difference). Pravastatin is a substrate of organic anion-transporting polypeptide (OATP) 1B1 and 1B3, and glecaprevir is an inhibitor of OATP1B1/3. Following coadministration with glecaprevir and pibrentasvir, increases in pravastatin exposure were similar to those observed when coadministered pravastatin was coadministered with clarithromycin (↑Cₘₐₓ to 2.1-fold, ↑AUC to 2.3-fold). When pravastatin is administered with glecaprevir and pibrentasvir, the dose of pravastatin should be reduced by 50%.

When coadministered with glecaprevir and pibrentasvir, rosuvastatin Cₘₐₓ and AUC24 were 5.6- and 2.2-fold, respectively, of rosuvastatin alone. Glecaprevir and pibrentasvir exposures were similar (≦25% difference) with and without rosuvastatin. Rosuvastatin is a substrate of breast cancer resistance protein (BCRP) and OATP1B1/3. Glecaprevir and pibrentasvir are inhibitors of BCRP, and glecaprevir is an inhibitor of OATP1B1/3. Increases in rosuvastatin exposure when coadministered with glecaprevir and pibrentasvir were similar to those seen when rosuvastatin was coadministered with lopinavir/ritonavir (↑5-fold Cₘₐₓ, 2.1-fold AUC). As per prescribing information, rosuvastatin dose should not exceed 10 mg QD when coadministered with lopinavir/ritonavir. Similarly, when rosuvastatin is coadministered with glecaprevir and pibrentasvir, the dose of rosuvastatin for adult patients should be limited to 10 mg QD.

### Example 5. Cyclosporine

Cyclosporine was evaluated at 100 mg and 400 mg in drug-drug interaction studies. Based on these study results, only subjects on stable doses of cyclosporine ≦100 mg per day should initiate the glecaprevir and pibrentasvir combination therapy, and cyclosporine dose may be adjusted according to normal therapeutic monitoring thereafter (e.g. may exceed 100 mg per day).

### Example 6. Drug-Drug Interaction Evaluation in Overseas Subject

The drug-drug interaction (DDI) of glecaprevir and pibrentasvir was characterized from Phase 1 clinical studies in overseas subjects.

DDI was evaluated between glecaprevir and pibrentasvir and drug transporter and CYP probes (CYP3A4, CYP1A2, CYP2D6, CYP2C9 and CYP2C19) and concomitant medications used in HCV-infected subjects including anti-retroviral (ARVs), calcium-channel blockers (CCB), angiotensin-receptor blockers (ARBs), opioids and tacrolimus. Safety and tolerability were assessed throughout the studies.

No clinically relevant changes were observed in CCBs, ARBs, opioids, ARVs and CYP probe substrates when coadministered with glecaprevir and pibrentasvir. Glecaprevir and pibrentasvir increased exposures of tacrolimus (˜↑45% AUC), pravastatin (↑2.3-fold AUC), rosuvastatin (↑2.2-fold AUC), dabigatran (↑2.4-fold AUC) and digoxin (↑48% AUC). No clinically significant changes were observed in GLE/PIB exposures. No serious adverse events were reported with these drugs.

Overall, glecaprevir and pibrentasvir has minimal clinical interaction with CYP and UGT enzymes, while exposures of OATP, P-gp and BCRP substrates may increase when used with GLE/PIB. No dose adjustment is recommended for CCBs, ARBs, opioids, tacrolimus and evaluated ARVs. Dose adjustment is recommended for sensitive substrates of OATP, P-gp and/or BCRP when coadministered with GLE/PIB. “Evaluated ARVs” includes to abacavir, cobicistat, dolutegravir, elvitegravir, emtricitabine, lamivudine, raltegravir, rilpivirine, or tenofovir alafenamide containing regimens.

The foregoing description of the present invention, including selected examples provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A method of treating patients infected with hepatitis C virus having an independent co-morbid condition, comprising co-administering to said patients glecaprevir and pibrentasvir once daily, together with a concomitant drug for treating the co-morbid condition, wherein said drug is selected from a group consisting of drugs that are substrates of Organic Anion Transporting Polypeptide, P-glycoprotein and Breast Cancer Resistance Protein, further wherein the established dose of said drug is dose-adjusted prior to administering glecaprevir and pibrentasvir to said patient.

2. The method of claim 1, wherein the glecaprevir is administered 300 mg once a day and the pibrentasvir is administered 120 mg once a day.

3. The method of claim 1, wherein the glecaprevir and the pibrentasvir is administered for a duration of 8, 12 or 16 weeks.

4. The method of claim 1, wherein said patient has a HCV genotype of 1, 2, 3, 4, 5 or 6.

5. The method of claim 1, wherein said patient is treatment naïve, treatment experienced or has cirrhosis.

6. The method of claim 1, wherein said drug is selected from a group consisting of digoxin, pravastatin, rosuvastatin, fluvastatin, pitavastatin and cyclosporine.

7. The method of claim 6, wherein the digoxin dose is reduced by 50% of the established dose.

8. The method of claim 6, wherein the pravastatin dose is reduced by 50% of the established dose.

9. The method of claim 6, wherein the rosuvastatin dose is no more than 10 mg per day.

10. The method of claim 6, wherein the cyclosporine dose is no more than 100 mg per day.

11. The method of claim 6, wherein the fluvastatin or pitavastatin dose is reduced to lowest approved dose or lowest necessary dose.

12. The method of claim 1, wherein administration of the glecaprevir and the pibrentasvir is not recommended or contraindicated for a concomitant treatment with a drug selected from a group consisting of atazanavir, rifampin, carbamazepine, hypericum perforatum, efavirenz, ethinyl estradiol containing medications, darunavir, lopinavir, ritonavir, atorvastatin, lovastatin and simvastatin.

13. A method of treating patients infected with hepatitis C virus having an independent co-morbid condition, comprising co-administering to said patients glecaprevir and pibrentasvir once daily, together with a concomitant drug for treating the co-morbid condition, wherein the established dose of said drug is dose-adjusted prior to administrating glecaprevir and pibrentasvir to said patient.

14. The method of claim 13, wherein the glecaprevir is administered 300 mg once a day and the pibrentasvir is administered 120 mg once a day.

15. The method of claim 13, wherein the glecaprevir and the pibrentasvir is administered for a duration of 8, 12 or 16 weeks.

16. The method of claim 13, wherein said patient has a HCV genotype of 1, 2, 3, 4, 5 or 6.

17. The method of claim 13, wherein said patient is treatment naïve, treatment experienced or has cirrhosis.

18. The method of claim 13, wherein said drug is selected from a group consisting of digoxin, pravastatin, rosuvastatin, fluvastatin, pitavastatin and cyclosporine.

19. The method of claim 18, wherein the digoxin dose is reduced by 50% of the established dose.

20. The method of claim 18, wherein the pravastatin dose is reduced by 50% of the established dose.

21. The method of claim 18, wherein the rosuvastatin dose is no more than 10 mg per day.

22. The method of claim 18, wherein the cyclosporine dose is no more than 100 mg per day.

23. The method of claim 18, wherein the fluvastatin or pitavastatin dose is reduced to lowest approved dose or lowest necessary dose.

24. The method of claim 13, wherein administration of the glecaprevir and the pibrentasvir is not recommended or contraindicated for a concomitant treatment with a drug selected from a group consisting of atazanavir, rifampin, carbamazepine, hypericum perforatum, efavirenz, ethinyl estradiol containing medications, darunavir, lopinavir, ritonavir, atorvastatin, lovastatin and simvastatin.

25. A method of treating patients infected with hepatitis C virus having an independent co-morbid condition, comprising co-administering to said patients glecaprevir and pibrentasvir once daily, together with a concomitant drug for treating the co-morbid condition, wherein the established dose of said drug is dose-adjusted prior to administrating glecaprevir and pibrentasvir to said patient, wherein said drug is selected from a group consisting of digoxin, pravastatin, rosuvastatin, fluvastatin, pitavastatin and cyclosporine.

26. The method of claim 25, wherein said drug dose-adjustment is selected from a group consisting of (a) the digoxin dose is reduced by 50% of the established dose, (b) the pravastatin dose is reduced by 50% of the established dose, (c) the rosuvastatin dose is no more than 10 mg per day, (d) the cyclosporine dose is no more than 100 mg per day, and (e) the fluvastatin or pitavastatin dose is reduced to lowest approved dose or lowest necessary dose.
